(19) United States
(12) Patent Application Publication
(10) Pub. No.: US 2017/0236397 A1
(43) Pub. Date: Aug. 17, 2017

(54) SAFETY CONTROL ROOM

(71) Applicant: Total Safety U.S., Inc., Houston, TX (US)

(72) Inventors: Dave Myslenski, Seabrook, TX (US); Bart Peetermans, League City, TX (US); Paul Tyree, Houston, TX (US)

(73) Assignee: Total Safety U.S., Inc., Houston, TX (US)

(21) Appl. No.: 15/518,513

(22) PCT Filed: Nov. 20, 2015
(86) PCT No.: PCT/US2015/061818

§ 371 Date: Apr. 12, 2017

Related U.S. Application Data
(60) Provisional application No. 62/082,493, filed on Nov. 20, 2014.

(51) Int. Cl. G08B 21/04; G01N 33/00; G08B 21/18; H04N 7/18

(52) U.S. Cl. CPC G08B 21/0453; H04N 7/183; G01N 33/0036; G08B 21/182

(57) ABSTRACT

A safety control system for a worksite includes a video system having a video camera communicable with a video monitoring device and a video recording device, a personnel system having a personnel sensor communicable with a personnel monitoring device and a personnel recording device, a component system having a component sensor communicable with a component monitoring device and a component recording device, and a reporting system configured to access at least one of the video recording device, the personnel recording device, and the component recording device. The reporting system generates a report including information from at least one of the video recording device, the personnel recording device, and the component recording device

### BACKGROUND

### Field of the Disclosure

Embodiments disclosed herein generally relate to a safety control room. More specifically, embodiments disclosed herein relate to safety control systems and methods for monitoring, reporting, and controlling personnel, components, access, and compliance, among others at facilities (e.g., refineries) and/or worksites.

### Related Art

In chemical, petrochemical, refining, and other industries, personnel may be exposed to harsh or extreme working conditions. Further, in such industries, in order to comply with local, state, and/or national regulations, industry operations must abide by particular compliance requirements when performing a standard operation (e.g., refinery shut down) or executing a non-standard task (e.g., emergency tasks). In order to ensure that personnel are adequately trained, healthy, and are operating safely when exposed to particular working conditions, personnel, components, access, and compliance at facilities, refineries, and/or production sites may be monitored, reported, and/or controlled.

### SUMMARY

In general, in one aspect, the present disclosure relates to a safety control system for a worksite including a video system having a video camera communicable with a video monitoring device and a video recording device, a personnel system having a personnel sensor communicable with a personnel monitoring device and a personnel recording device, a component system having a component sensor communicable with a component monitoring device and a component recording device, and a reporting system configured to access at least one of the video recording device, the personnel recording device, and the component recording device, in which the reporting system generates a report including information from at least one of the video recording device, the personnel recording device, and the component recording device.

In general, in another aspect, the present disclosure relates to a personnel monitoring system including a personnel sensor configured to sense attributes of a worker at a worksite, in which the personnel sensor is communicable with a personnel monitoring device and a personnel recording device, and in which the attributes of the worker comprise at least one of heart-rate, temperature, breathing air, surrounding gases, training, and fatigue.

In general, in yet another aspect, the present disclosure relates a compliance monitoring system including a compliance sensor communicable with a compliance monitoring device and a compliance recording device, in which the compliance monitoring system is configured to compare data from the compliance sensor with compliance requirements, and in which the compliance requirements are worksite specific.

Other aspects and advantages of the disclosure will be apparent from the following description and the appended claims.

### BRIEF DESCRIPTION OF DRAWINGS

FIGS. 1A-1C depict a safety control system in accordance with one or more embodiments of the present disclosure.

FIG. 2 depicts a confined workspace in accordance with one or more embodiments of the present disclosure.

FIG. 3 depicts a restricted area in accordance with one or more embodiments of the present disclosure.

FIGS. 4A-4H depict a breathing air system in accordance with one or more embodiments of the present disclosure.

FIGS. 5A-5D depict personnel monitoring in accordance with one or more embodiments of the present disclosure.

FIGS. 6A-6C depict a central station in accordance with one or more embodiments of the present disclosure.

### DETAILED DESCRIPTION

Specific embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the invention. However, it will be apparent to one of ordinary skill in the art that the embodiments disclosed herein may be practiced without these specific details. In other instances, well-known features have not been described in detail to avoid unnecessarily complicating the description.

Furthermore, those having ordinary skill in the art will appreciate that when describing a first element to a second element disposed thereon, it is understood that disposing may be either directly disposing the first element on the second element, or indirectly disposing the first element on the second element. For example, a first element may be directly disposed on a second element, such as by having the first element and the second element in direct contact with each other, or a first element may be indirectly disposed on a second element, such as by having a third element, and/or additional elements, disposed between the first and second elements.

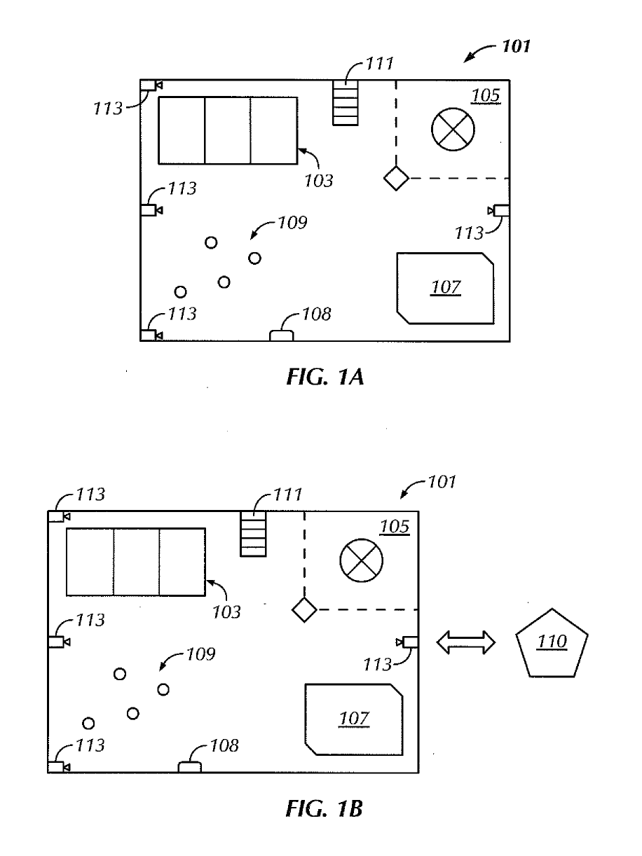

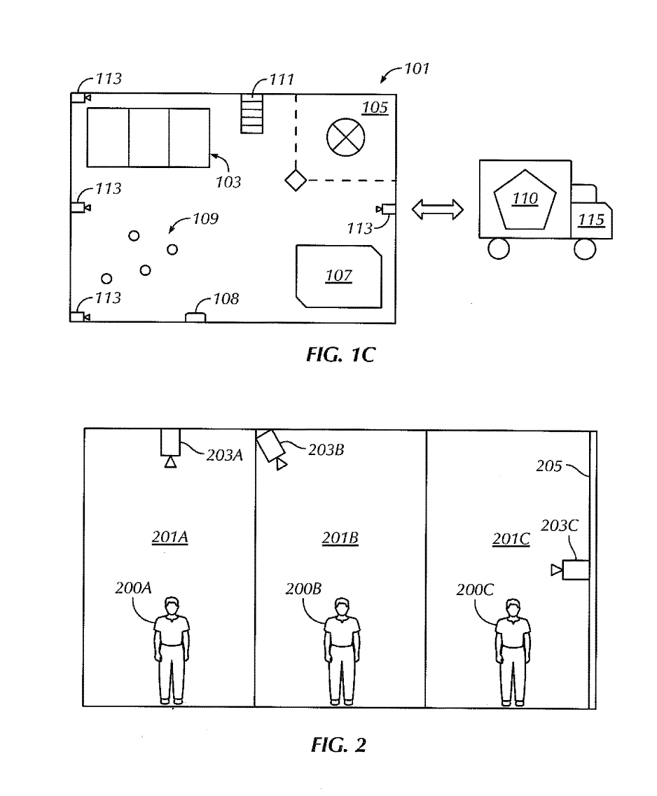

As shown in FIGS. 1A-1C, a safety control system in accordance with one or more embodiments of the present disclosure is shown. In one or more embodiments, one or more of the elements shown in FIGS. 1A-1C may be omitted, repeated, and/or substituted. Accordingly, the present disclosure should not be considered limited to the specific arrangements of elements shown in FIGS. 1A-1C.

In one or more embodiments, a safety control system may monitor, record, control, and/or report operations, working conditions (environmental conditions such as weather, area conditions, etc.), component status (active, inactive, damaged, inoperable, etc.), compliance regulations (emergency procedures, number of personnel per area, safety validation of one or more components, etc.), and personnel (employees, workers, contractors, etc.) status (vitals such as heart rate, training experience, authorized access, etc.), among many others, at a worksite 101.

The worksite 101 may be a building, such as factory having multiple floors or levels with different operations being performed by personnel throughout the building. The worksite 101 may be a remote worksite, such as an oilfield, with personnel working outside that may experience or be exposed to different environmental conditions throughout the duration of a project and where a number of different tasks are performed in different areas of the oilfield. Further, the worksite 101 may be a facility that includes both indoor and outdoor working conditions, such as a refinery or petrochemical plant, with personnel that may be exposed to harsh working conditions, such as confined spaces or toxic fumes. As such, although the worksite 101 has been described with respect to the examples above, the present disclosure is not so limited. The worksite 101 may include or refer to any environment where work is being performed which may or may not be performed by active personnel. For example, the worksite 101 may include several components performing automated tasks where personnel may not be necessary.

In one or more embodiments, as shown in FIGS. 1A-1C, the worksite 101 may include a confined workspace 103, a restricted area 105, a breathing air system 107, and an open workspace 109. As shown, the safety control system may include a central station 110 for monitoring, recording, controlling, and/or reporting working conditions, component status, compliance regulations, and personnel status, among many others, at the worksite 101. The central station may be permanently located at the worksite 101 or the central station 110 may be mobile. For example, as shown in FIG. 1C, the central station 110 may be disposed on a vehicle 115, and may be movable to one or more worksites. Accordingly, the central station 110 may be configured and setup for monitoring, recording, controlling, and/or reporting working conditions, component status, compliance regulations, and personnel status, at any worksite known in the art and may be transported between worksites or moved to different locations within a worksite 101.

The worksite 101 may include a number of floors or levels accessible using stairs 110. Although not shown, the number of floors or levels may be accessible using any means known in the art, such as an elevator, ladder, or lift, for example. The worksite 101 may also include a personnel station 108 where one or more personnel may check out equipment, obtain permits or access badges, view maintenance logs, etc.

As shown in FIGS. 1A-1C, the worksite 101 may include an audio visual (AV) system having one or more video cameras 113 equipped with audio devices (e.g., microphones and/or speakers). The one or more video cameras 113 may communicate with an AV monitoring device and an AV recording device. The AV monitoring and recording devices may be located at the central station 110 or may be located remotely, on-site, or connected directly or near to the one or more video cameras 113. The AV system may monitor and/or record operations in any or all areas of the worksite 101 including, for example, confined workspace 103, restricted area 105, breathing air system 107, and open workspace 109. In addition, the AV system may monitor and/or record operations about the perimeter of the worksite 101 or about a perimeter of any area of the worksite 101 to detect unauthorized users and/or prevent unauthorized personnel from entering the worksite or a specific area of the worksite 101.

In one or more embodiments, the AV system may feed live video and audio to the AV monitoring device and the AV recording device. As discussed herein, live feed video and audio refers to distribution of video and audio data in real time or at a slight delay in real time. As such, real time video monitoring may allow for immediate feedback to unsafe working conditions. For example, the AV system may also be capable of visual and audio alerts given certain conditions by blinking lights or sounding a siren in an emergency situation, such as a fire, flood, or natural disaster. In addition, the AV system may also be used to monitor personnel conditions and workspace environments. For example, if one or more personnel are in danger (e.g., if the personnel are exposed to toxic gases), emergency services may be dispatched and the AV system may indicate (e.g., using lights and/or sirens, for example) the location of the personnel in danger. Thus, emergency services personnel would more easily be able to find, rescue, and tend to the one or more personnel that may be in danger.

Further, the AV recording device may be used to store video and audio data for future reference. In doing so, the video and audio data may be used for on-the-job training, lesson learning, or off site classroom lectures.

As many tasks may benefit from constant and live monitoring, the AV system may be linked or connected to (wired or wirelessly) the central station 110. As such, the number of personnel that are needed to monitor a particular task or worksite may be reduced. In addition, personnel, worksites, and/or operations may be monitored and/or controlled at a single location on-site and/or remotely from the central station 110. For example, one or more video cameras 113 may be controlled by one or more users at the central station 110.

Referring now to FIG. 2, a confined workspace in accordance with one or more embodiments of the present disclosure is shown. In one or more embodiments, one or more of the elements shown in FIG. 2 may be omitted, repeated, and/or substituted. Accordingly, the present disclosure should not be considered limited to the specific arrangements of elements shown in FIG. 2.

In one or more embodiments, a worksite, such as worksite 101 in FIGS. 1A-1C, may include confined workspaces. Confined workspaces may include any space that is not normally accessible or not usually intended as workspaces in which one or more workers operate. Confined spaces may include boilers, tanks, tubing systems, ship holds, sealed off spaces, among many others known in the art. Because the space in a confined workspace is often limited or the workspace is difficult to access, operating within such a confined space is a potentially dangerous task. As such, a number of measures are taken to ensure that workers operating in confined spaces are healthy, performing their work safely, and not in danger.

As mentioned above, in some cases, workers may operate in environments with under such harsh conditions that the worker (and workspace) is sealed off from the rest of the worksite. For example, when the level of harmful gases are above a particular threshold, the area or workspace may be sealed off in order to prevent the harmful gases from being exposed to other personnel at the worksite. In FIG. 2, workers 200A-200C are shown working in confined workspaces 201A-201C, respectively. As it may not always be possible to directly oversee the safety of the workers 200A-200C by a safety officer, for example, when the confined workspaces 201A-201C are sealed, monitoring devices 203A-203C may be setup to monitor operations within the confined workspaces 201A-201C. Monitoring devices 203A-203C may be positioned at any location (fixed or movable) within or looking into the confined spaces 201A-201C. For example, monitoring device 203A is positioned at a top portion of confined space 201A viewing operations from above, while monitoring device 203B is positioned at a top portion of confined space 201B, but viewing operations at an angle.

Further, monitoring devices 203A-203C may be movable within confined spaces 201A-201C. For example, monitoring device 203C is disposed along a track 205 which allows monitoring device 203C to move along a length of confined space 201C. Monitoring device 203C may be configured to track movement of worker 200C or may be configured to view a particular area within confined space 201C and thus, may move with respect to worker 200C such that the monitoring device 203C maintains view of the particular area. In addition, monitoring devices 203A-203C may be capable of panning, tilting, zooming, focusing, as well as any other camera functionality known in the art.

In one or more embodiments, monitoring devices 203A-203C may include a video camera equipped with audio devices (e.g., microphone and a speaker) that may feed live video and/or audio to one or more monitoring and/or recording devices, such as those described above with respect to the AV system in FIGS. 1A-1C. The video and audio data may be transmitted wirelessly or wired to a central station, such a central station 110, as described above in FIGS. 1A-1C.

In addition, monitoring devices 203A-203C may also include a number of detectors to monitor the levels of one or more gases in each of confined spaces 201A-20C. Alternatively, gas detectors may be separate from monitoring devices 203A-203C as may be required by compliance regulations. For example, gas detectors may be positioned at particular areas of interest within confined spaces 201A-201C in order to detect certain gases based on density, temperature, and/or location within the confined space, among others. Further, monitoring devices 203A-203C may include other sensors or measuring devices (not shown) capable of monitoring the status of the workers 200A-200C and other workspace conditions, such as the temperature, breathing air (quality and volume), and any other workspace condition.

Further, in one or more embodiments, monitoring devices 203A-203C may be capable of multi-way communication. For example, monitoring devices 203A-203C may be equipped with microphones, speakers, and/or display devices for communicating with one or more personnel outside of confined spaces 201A-201C. For example, monitoring devices 203A-203C may be equipped with one or more displays for video conferencing with personnel outside of the confined spaces 201A-201C. Video conferencing may be useful when reaching a point during a particular task in which a worker may encounter an unexpected issue. For example, when welding within a confined space, a worker may accidentally damage one or more components that were to be welded. Thus, using the monitoring devices 201A-201C, a worker may communicate with personnel to discuss how to proceed or whether the task currently being performed should be abandoned. Although not shown, additional or alternative monitoring devices may be disposed on one or more workers 200A-200C. For example, a worker may be equipped with a video camera and a microphone capable of feeding live video and audio to personnel outside of the confined workspace. The equipment may be positioned such that the operation may be viewable from a direct perspective.

Further, any of monitoring devices 203A-203C may be used as redundant monitoring. For example, monitoring devices 203A-203C may transmit data to more than one location outside confined workspaces 201A-201C. Thus, a number of personnel at different locations may monitor operations in the confined workspaces 201A-201C.

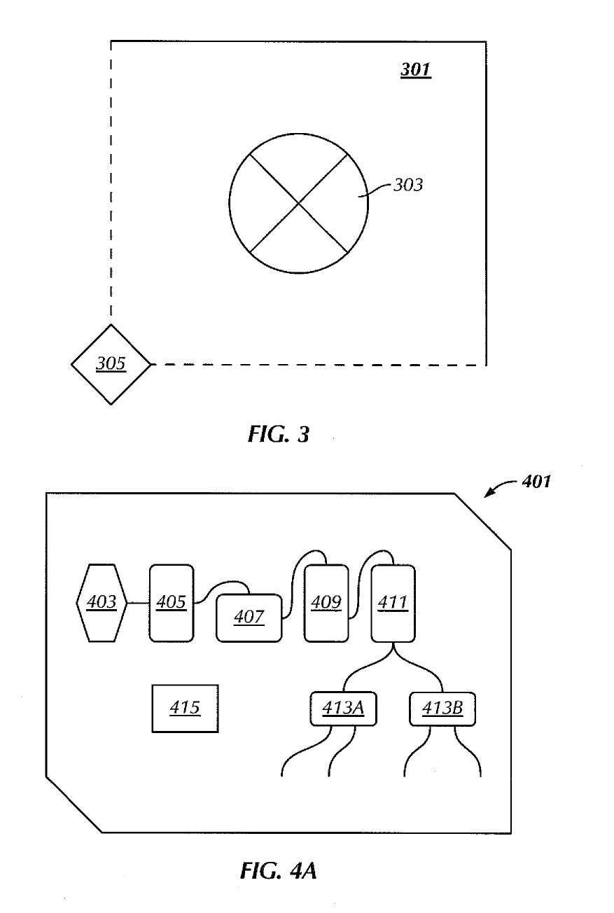

Referring now to FIG. 3, a restricted area in accordance with one or more embodiments of the present disclosure is shown. In one or more embodiments, one or more of the elements shown in FIG. 3 may be omitted, repeated, and/or substituted. Accordingly, the present disclosure should not be considered limited to the specific arrangements of elements shown in FIG. 3.

In one or more embodiments, access to an area of a worksite, such as worksite 101 in FIGS. 1A-1C may be limited. For example, one or more areas of a worksite may have restricted access due to operations being performed in the area. Other areas may have access restrictions based on training or confidentiality. For example, an area may be accessible only by employees having an up to date training record, a given confidential status, or particular training completed. In addition, certain areas of a worksite may be designated as danger zones where operations or conditions in such a danger zone is off limits to all personnel or only accessible by emergency services.

As shown in FIG. 3, a restricted area 301 includes a lift 303. The lift 303 may be operable by one or more personnel having adequate training, such as lift operators. As such, restricted area 301 may only be accessible by lift operators. One or more employees may gain access to restricted area 301 using access control unit 305. Access control unit 305 may be communicable with a central station, such as central station 110, as described above. In addition, the access control unit 305 may be programmable or controllable from a remote location or on-site. The access control unit 305 may be capable of defining a perimeter about the lift 303, such that only lift operators may access the area defined by the access control unit. Alternatively, the restricted area 305 may be predefined by personnel and may be modified based on operations performed within the restricted area 301.

The access control unit 305 may include information relating to training records of personnel, confidentiality status of personnel, as well as any other information relating to accessibility known in the art. In addition, the access control unit 305 may include a reader to be used by personnel attempting to gain access to restricted area 301. A personnel badge may be obtained by a worker, for example, by personnel station 108 in FIGS. 1A-1C, and may be scanned by the reader of the access control unit 305. The reader may scan the personnel badge using Radio-frequency identification (RFID), Bluetooth, magnetic strip, integrated circuit card, or any other scanning/reading technique known in the art.

Although not shown, in one or more embodiments, restricted area 301 may include any number of detectors, sensors, or AV system devices, as discussed above, and may be communicable with a central station. As such, the restricted area 301 may be monitored, such as when a critical or important operation is being performed (by lift 303 for example), by one or more personnel to prevent and/or manage safety of the operations performed in the restricted area 301. Similarly, the access control unit 305 may be controllable by one or more personnel. Alternatively, or in addition, the access control unit 305 may access information used for restricting access to restricted area 301.

In one or more embodiments, the restricted area 301 may be designated as a danger zone. As mentioned briefly above, a danger zone may be designated when a limited number of personnel, such as only emergency personnel, are allowed access to the restricted area 301. Accordingly, in one or more embodiments, the restricted area 301, when designated as a danger zone, may be capable of alerting personnel of the danger zone designation. For example, if toxins were spilled or a high risk operation is being performed in the restricted area 301, when personnel approach the restricted area 301, an alarm (visual or audio) may be triggered to alert the approaching personnel.

The approaching personnel may be recognized using cellular, Global Positioning System (GPS), or Wi-Fi, among others by detecting a personnel sensor (describe in detail below) carried by the approaching worker. In some scenarios, the designation of a danger zone may include an area bound by GPS coordinates and thus, when personnel approach or cross into the restricted area 301, an alarm (visual or audio) on the personnel sensor carried by the approaching worker may also be triggered. In other embodiments, if the restricted area 301 is designated a danger zone, the access control unit 305 may account for the designation by not allowing personnel that would have otherwise gained access to the restricted area 301 had the danger zone not be designated. Further, in one or more embodiments, an alarm for a danger zone designated area may also be triggered by motion detection, infrared, or any other detection technique known in the art.

Further, in one or more embodiments, restricted area 301 may limit personnel access based on electronic permitting. For example, one or more permits may be required by local, state, and/or federal regulations when performing a particular task at a worksite. In some cases, the number of personnel accessing the restricted area 301 at a given time may be limited, the amount of time personnel are working in the restricted area 301 may be limited, and the restricted area 301 may be entirely off limits to all personnel until a valid operating permit is obtained. Similar to the above, the access control unit 305 may access electronic permitting information stored on-site or remotely on a server accessible by a network, cellular communications, radio communications, Wi-Fi, or any other methods known in the art.

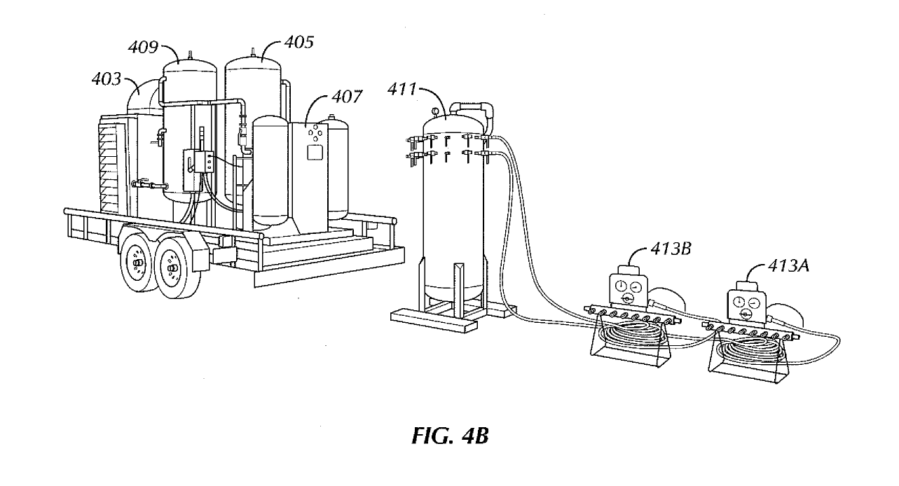

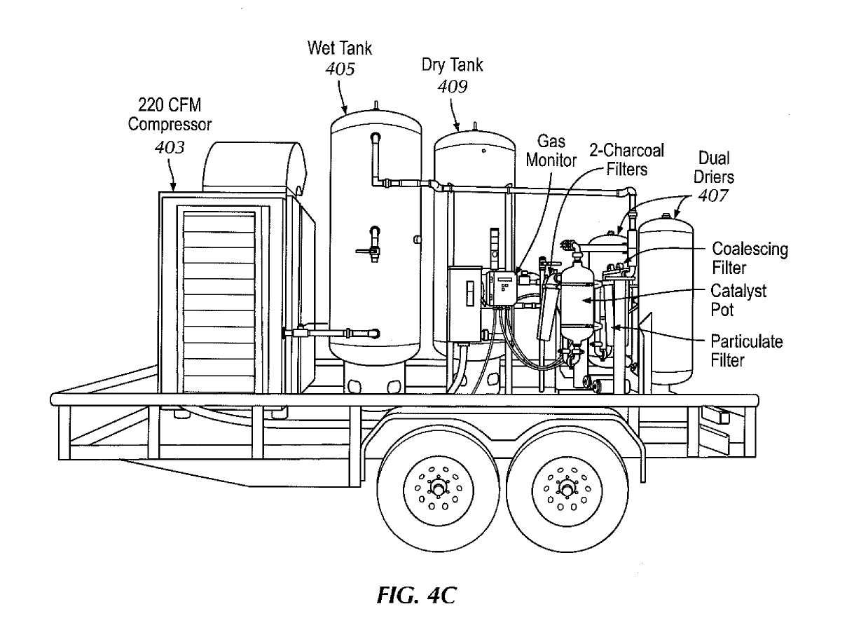

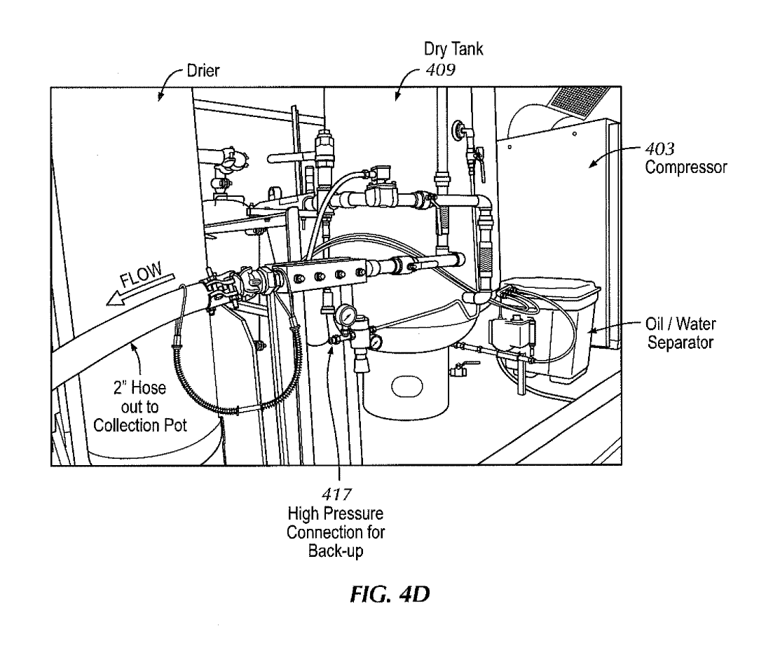

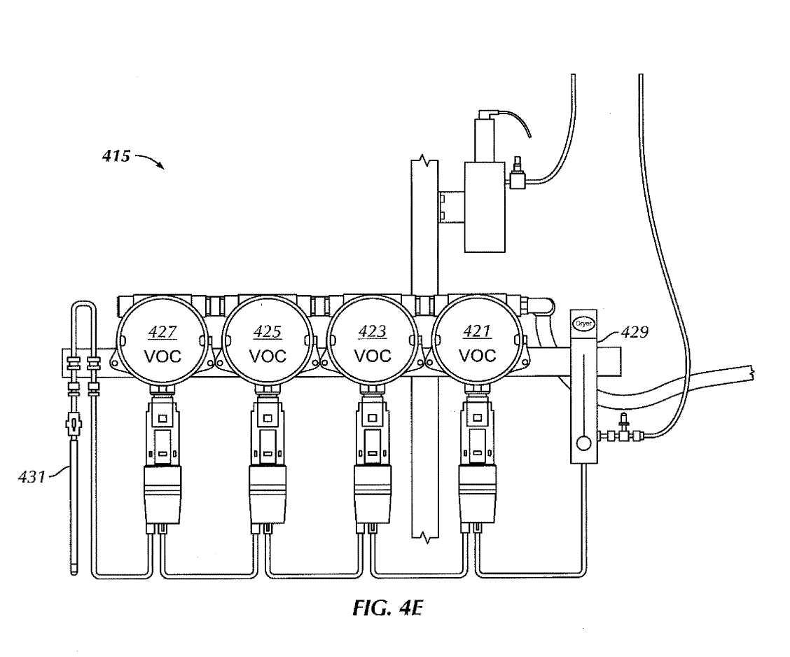

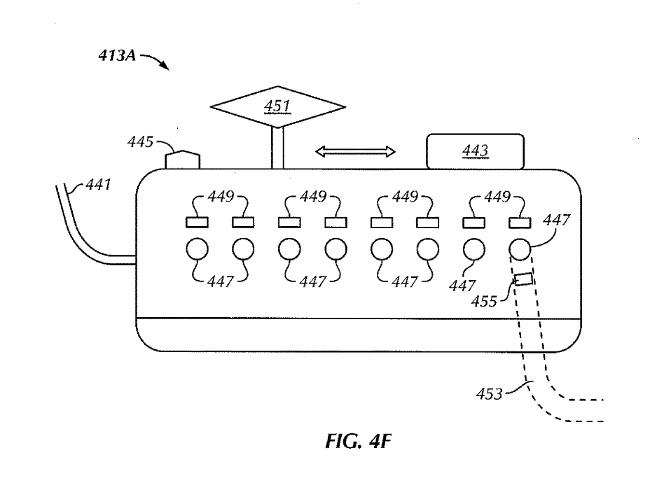

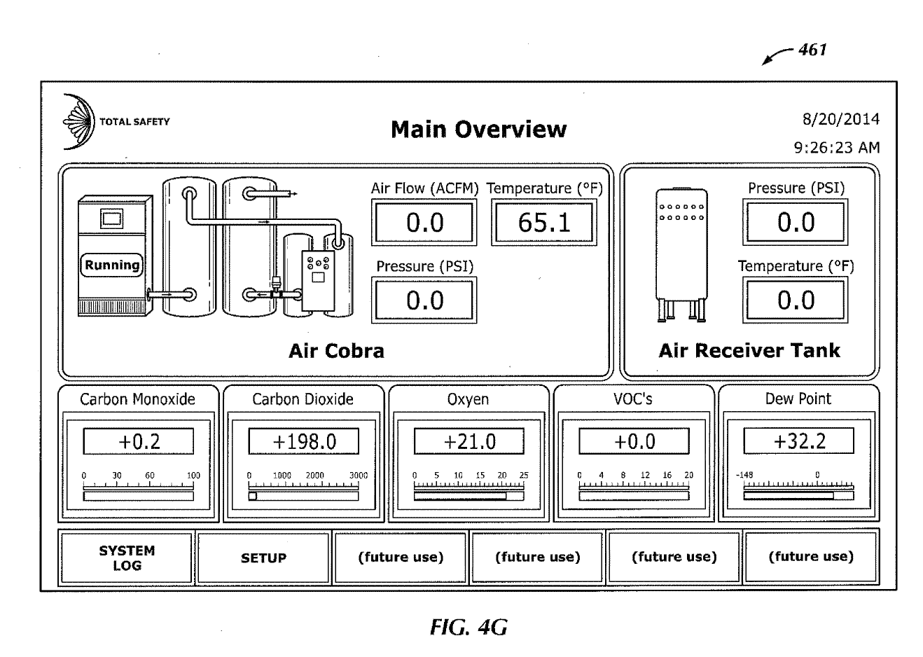

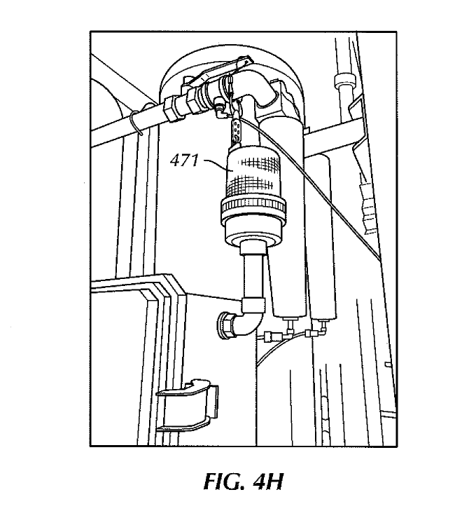

Referring now to FIGS. 4A-4H, a breathing system in accordance with one or more embodiments of the present disclosure is shown. In one or more embodiments, one or more of the elements shown in FIGS. 4A-4H may be omitted, repeated, and/or substituted. Accordingly, the present disclosure should not be considered limited to the specific arrangements of elements shown in FIGS. 4A-4H.

In one or more embodiments, airborne pollutants or contaminants may affect the ambient air in a given area of a worksite, such as worksite 101 in FIGS. 1A-1C. Such contaminants may be harmful if inhaled or may be generally dangerous to any personnel exposed to the contaminants. Accordingly, in such circumstances, the worksite may include an area having a breathing air system capable of producing and distributing breathable air to workers that would otherwise be exposed to breathing in the aforementioned pollutants and/or contaminants. In FIGS. 4A-44D, a breathing system 401 includes an air compressor 403 for receiving ambient air. The air compressor 403 is capable of the intake of surrounding air and distributing the intake air to wet tank 405. Although not shown, the air compressor 403 may include an air intake sensor configured to measure the intake air. The air intake sensor may measure percentages of different gases in the intake air, volume and type of particles contained with the intake air, and quality of the intake air.

As shown, wet tank 405 is communicable with purifier 407. The purifier 407 is capable of purifying the intake air by removing contaminants, moisture, oil vapor, among others, in order to generate breathing air for personnel. The purifier 407 may include a number of filters, such as a coalescing filter, charcoal filters, and a particulate filter, as shown in FIG. 4C. Purifier 407 is communicable with dry tank 409 which distributes the purified breathing air to a collection pot 411. Although only one collection pot 411 is shown, dry tank 409 may distribute breathing air to a plurality of collection pots. Dry tank 409 may include a high-pressure connection 417 for connecting to a reserve air supply, such as a high-pressure tube trailer (not shown).

The Occupational Safety and Health Administration (OSHA) requires a particular quality of breathing air for use in working environments. For example, Grade D breathing air has been adopted by OSHA as the standard for breathing air at a worksite. Grade D breathing air requires a number of filtration specifications, pressures, and levels of specific gases and moisture content. Accordingly, in order to comply with OSHA standards, after purification, breathing air may be monitored using gas monitor 415, as shown in FIGS. 4C and 4E.

As shown in detail in FIG. 4E, gas monitor 415 is configured to measure the breathing air prior to being distributed to the collection pot 411. Gas monitor 415 includes an oxygen sensor 421 for measuring the oxygen content of the purified breathing air. Gas monitor 415 also includes a carbon dioxide sensor 423 and a carbon monoxide sensor 425 for measuring carbon dioxide content and the carbon monoxide content, respectively, of the purified breathing air. In addition, gas monitor 415 includes volatile organic compound sensor 427 and a visual flowmeter 429 for measuring flow rate. Further, gas monitor 415 also includes a condensed oil monitor 431 for monitoring condensed oil content.

Referring back to FIGS. 4A-4D, collection pot 411 further distributes the breathing air to manifolds 413A and 413B. Manifolds 413A and 413B are shown in further detail in FIG. 4F. As shown in FIG. 4F, a manifold 413A includes an intake hose 441 from collection pot 411, as shown in FIGS. 4A-4D. Manifold 413A also includes a battery 443, a control sensor 445, outlets 447, and hose sensors 449. The battery 443 powers a number of sensors of the manifold 413A, such as control sensor 445 and hose sensors 449. In addition, the battery 443 may be chargeable using solar panel 451. Alternatively, solar panel 451 may directly power one or more sensors of the manifold 413A thus enabling the manifold 413A to be placed at a location far from a power source.

The control sensor 445 is configured to measure and detect incoming flow rates and pressures in hose 441 from collection pot 411. The control sensor 445 also detects battery discharging rate and power level remaining before manifold 413A requires recharging. Control sensor 445 also may activate or deactivate power to or from each of the sensors 449, battery 443, and solar panel 451. As such, control sensor 445 may be controllable or monitored at a remote location, such as central station 110, as described above. Control sensor 445 may also include GPS information and may communicate with a central station via Wi-Fi, radio communication, internet, or any other communication method known in the art.

In one or more embodiments, a user may connect a hose 453 to an outlet 447 of the manifold 413A such that breathing air is distributed from the collection pot 411 through manifold 413A to an end user (not shown) using hose 453. The hose 453 may include a hose sensor 455 detectable by the outlet sensor 449. The outlet sensor 449 may detect hose sensor 455 via RFID, Bluetooth, Wi-Fi, near-field communication, or any other method known in the art and may communicate information (wired or wirelessly) to the control sensor 445 and/or one or more monitoring devices at a central station. The hose sensor 455 may include information using an integrated circuit chip, flash storage, magnetic strip information, or any other data storage device known in the art communicable with the outlet sensor 449 and/or the control sensor 445. The hose sensor 455 may include information with respect to hose 453 and/or the end user using the hose 453. Information such as maintenance requirements including dates, times, and authorized users that last checked hose pressure, cleaned the hose, and used the hose, for example. Other information may include the flow rate through the hose 453 at a given time.

The hose sensor 455 may also indicate to outlet sensor 449 that a user is connected to a particular outlet and may be activated using a contact switch (not shown). In one or more embodiments, the hose 453 may be attached using a locking mechanism (not shown). For example, the hose 453 may engage with an electromagnetic lock that is unlockable using a badge or swipe card.

Referring to FIG. 4G, in one or more embodiments, the breathing air system 401 may be monitored and controlled using an interface communicable with any of the sensors described above. For example, as shown in FIG. 4G, an interface 461 may include a number of displays and may receive input via an input device. In other embodiments, the interface 461 may be touch screen. As shown, the interface 461 is configured to monitor and control a number of aspects of the breathing air system 401. Specifically, the interface 461 may monitor and/or control the air flow, temperature, and pressure at any point along the production of breathing air using sensors in direct or indirect communication with the breathing air system 401. In addition, the interface may monitor the output or input air and may include monitoring such as carbon dioxide and oxygen content in the produce breathing air, as described above.

The interface 461 may also monitor and/or control sensors of the manifold.

For example, the interface 461 may monitor the number of users on a given manifold and the pressure distributed to each manifold from one or more collection pots. The interface 461 may also activate or deactivate one or more components of the breathing air system 401. For example, if the quality of air is not up to standards, the compressor 403 may be halted and production of breathing air may cease until the problem is resolved.

Referring to FIG. 4H, in one or more embodiments, the breathing air system 401 may include an alarm to be triggered in an emergency situation. The alarm may include one or more visual alarms, such as light 471, and may include one or more audio alarms (not shown). The alarm may be triggered remotely from a central station or may be triggered in response to a production issue. For example, if the quality of breathing air falls below the Grade D standard, an alarm may be triggered to inform the personnel operating the breathing system of the issue.

Although a number of examples of monitoring and control have been described above, those having ordinary skill in the art would appreciate that any number of components, systems, and parameters of the breathing air system may be monitored and controlled using any interface known in the art and may be monitored and/or controllable from any location using cellular, radio, or any other communication methods known in the art.

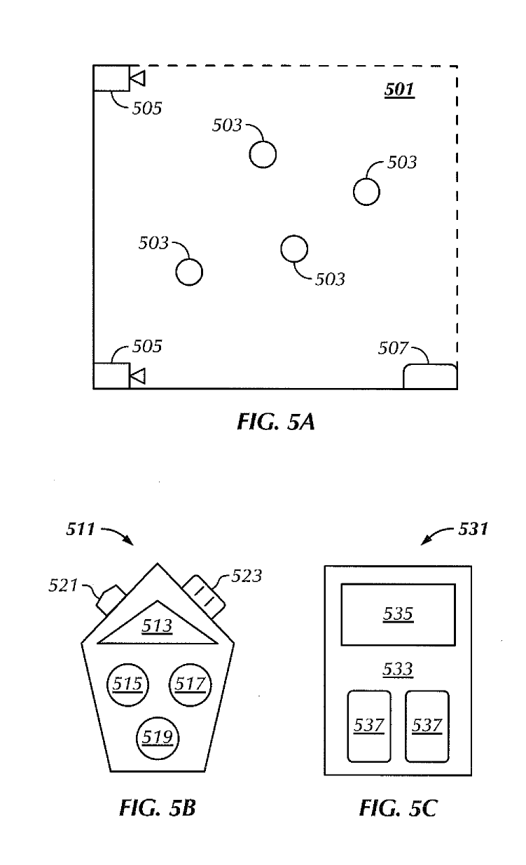

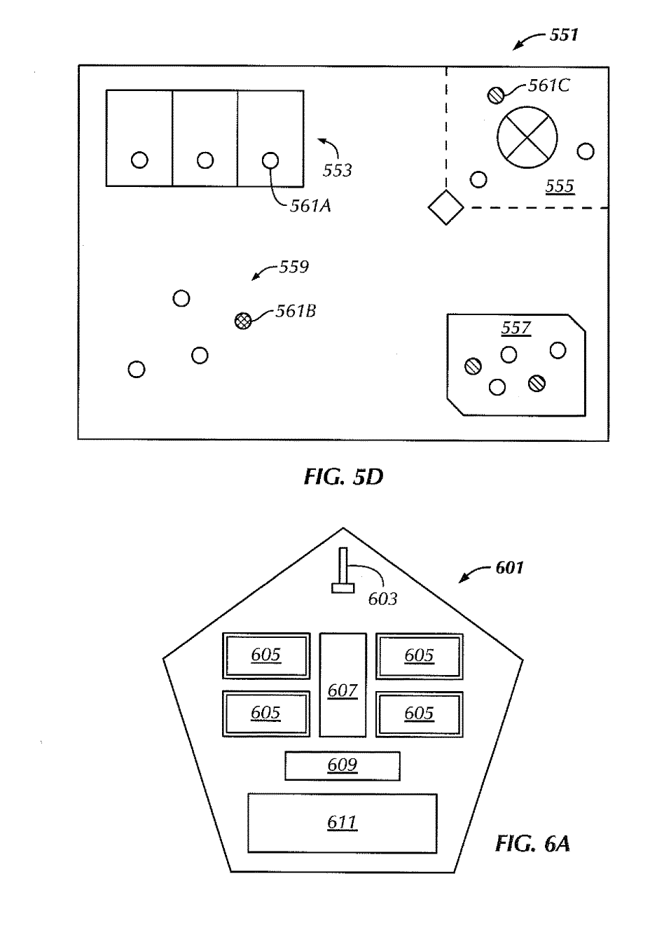

Referring now to FIGS. 5A-5D, an open workspace in accordance with one or more embodiments of the present disclosure is shown. In one or more embodiments, one or more of the elements shown in FIGS. 5A-5D may be omitted, repeated, and/or substituted. Accordingly, the present disclosure should not be considered limited to the specific arrangements of elements shown in FIGS. 5A-5D.

In one or more embodiments, a worksite, such as worksite 101 in FIGS. 1A-1C, may include a number of open workspaces open to the elements. As such, open workspaces may present a number of working hazards and/or environmental working conditions that may be dangerous for personnel. In FIG. 5A, a top down view of an open workspace 501 is shown. The workspace 501 includes a number of workers 503 operating and being exposed to environmental and facility specific risks. To monitor safety, monitoring devices 505 may be used and may include AV devices, such as those described above. The AV devices may be used to monitor and/or alert workers while operating and may be communicable with a central station, such as central station 110. In addition, the workspace 501 includes a personnel station 507. The personnel station may be used by the workers 503 to check out equipment, obtain permits, and access training materials, among others.

To monitor their safety, workers 503 may be equipped with a personnel sensor. Referring now to FIG. 5B, a personnel sensor 511 is shown. The personnel sensor 511 includes a display 513 for viewing information. The personnel sensor 511 also includes a number of sensors, such as temperature sensor 515 and gas detector 517, and may also include storage, such as memory 519. The personnel sensor 511 may also include a control sensor 521 and an alarm 523. Although a number of sensors and indicators are described above, any number and type of sensors may be included in the personnel sensor without departing from the scope of the present disclosure.

In one or more embodiments, sensor information from at least one of the temperature sensor 515 and gas detector 517, for example, may be displayed on display 513. Display 513 may also include information relating to current GPS coordinates, emergency statuses in the facility, danger zone designations. The personnel sensor 511 may include additional monitoring devices such as a microphone, speaker, and a camera, for example.

In addition, the personnel sensor 511 may be connected (wired or wirelessly) to a number of other sensors used to measure vitals of a worker. For example, the personnel sensor 511 may be connected to a device for monitoring biometrics of the worker, such as the heart-rate or temperature of the worker. This information may be displayed using display 513. Further, the personnel sensor 511 may be communicable with and controllable by a central station, such as central station 110. As such, the central station may be capable of triggering the alarm 523 under certain circumstances. For example, if the vitals of a worker fall outside a particular range, and the worker passes out, the alarm may be triggered remotely and emergency services may be dispatched knowing the workers location via GPS. The alarm may blink or sound further aiding the emergency service personnel to the location of the worker.

Referring now to FIG. 5C, another embodiment of a personnel sensor is shown. In this example, personnel sensor 531 may include a badge 533 having identification information 535 printed thereon. The identification information 535 may include worker identification, training completion, confidentiality status, known diseases or allergies, and any other information known in the art. Personnel sensor 531 may also include a RFID chip 537 and an integrated circuit chip 539. The RFID chip 537 may be configured to be accessible using a particular radio frequency and may include any identification information or additional information. Similarly the integrated circuit chip 539 may also include identification information scannable or detectable by one or more reader devices. The badge 533 may also be designed to measure clock-in and clock-out hours over time. As such, the number of hours worked by a worked may be stored on the RFID chip 537 and/or the integrated circuit chip 539 and may be used to manage fatigue of a worker. For example, if a worker works more than a specific number of hours over a given period of time (e.g., 2 days), the worker may be more likely to be tired and perform inadequately or unsafely. Therefore, personnel devices, such as personnel sensors 511 and 531 may be used to prevent hazards caused by fatigued or overworked workers.

Further, a worker may be equipped with both personnel sensors 511 and 531 that may communicate with each other. For example, badge 533 may include an element detection device, such as a device configured to measure exposure to radiation, and may communicate the detection to personnel device 511 which may be displayed on display 513. Therefore, unnecessary or overexposure to harmful gases or radiation may be known by the worker as soon as the worker has exceeded the recommended levels of exposure and before health issues may arise.

In addition, each of the personnel sensors 511 and 531 may be communicable with a central station and may store and/or transmit data to recording devices. Thus, a data log of personnel work history, exposure, and health can be monitored, stored, and analyzed to enhance safety at worksites.

As discussed above, personnel sensors may include GPS and/or other location information. In addition, personnel sensors may include or have access to health information, training information, and any other information known in the art. As such, the location and status of personnel at a worksite may be monitored during operation on site or from a central station.

Referring now to FIG. 5D, a worksite 551 is shown having a confined workspace 553, a restricted area 555, a breathing air system 557, and an open workspace 559 and is monitored from a central location. As shown, workers are indicated by circles and are distributed and operating in different location through the worksite 551. For example, worker 561A is operating within confined workspace 553, worker 561B is operating in open workspace 559, and worker 561C is operating in restricted area 555. Thus, using personnel sensors in communication with monitoring and/or recording devices at a central location, the position and status of each work may be shown and/or displayed on a monitoring device.

In one or more embodiments, the status of each worker may be indicated and/or monitored. In this example, the status of each worker is indicated by the shade of the circle representing each worker. No shade (i.e., white circle) indicates that the worker is healthy and all training is adequate for the work that is being performed. As shown, worker 561A operating in confined space 553 and is healthy with no immediate risk indicated.

Gray shade indicates a non-immediate risk. For example, the worker may be exposed to higher than normal operating conditions but the conditions are still suitable for working. In other instances, a worker may have all of the necessary training, but the training is due to expire later this month. As shown, worker 561C is shaded gray. Thus, worker 561C may be exposed to higher temperatures than desired for ideal working conditions, but not high enough to warrant immediate action. In general, gray shaded workers are suitable for working, but may soon be of concern.

Black shade indicates that the worker is at risk and the risk is of immediate concern. For example, an employee may be in an unauthorized area of the worksite or that training certificates for the work being performed have expired. As shown, worker 561B is shaded black and thus, may not be adequately trained to perform the task or work in the open workspace 559. Although only grayscale shading has been described above, any color and any number of colors may be used to indicate status of personnel at a worksite. For example, training expirations may be shaded a certain color, while high-temperature or abnormal heart-rate may be shaded a different color. Those having skill in the art would appreciate that any number of status indicators may be realized without departing from the scope of the present disclosure.

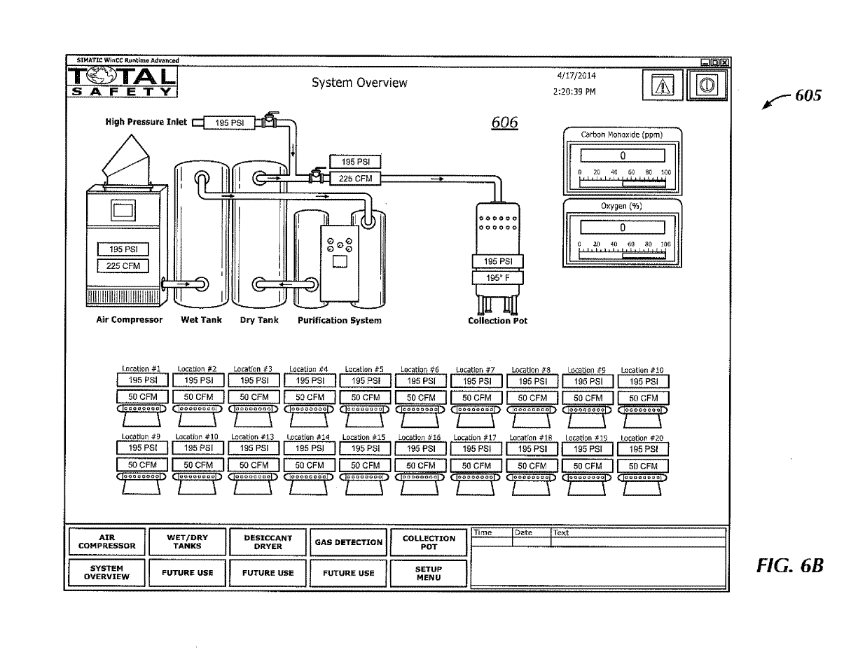

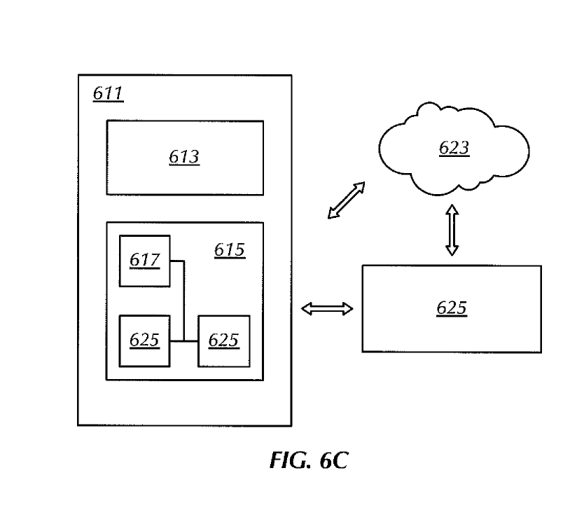

As shown in FIGS. 6A-6C, a central station in accordance with one or more embodiments of the present disclosure is shown. In one or more embodiments, one or more of the elements shown in FIGS. 6A-6C may be omitted, repeated, and/or substituted. Accordingly, the present disclosure should not be considered limited to the specific arrangements of elements shown in FIGS. 6A-6C.

As described above, a number of components, systems, and personnel may be in communication with a central station. As shown in FIG. 6A, a central station 601 includes an antenna 603, monitoring devices 605, a compliance monitor 607, a recording device 609, and a control device 611. The antenna 603 is configured to transmit and receive signals to and/or from a worksite, such as worksite 101 in FIGS. 1A-1C. As described above, the antenna 603 may receive signals from worksite monitoring devices, access control units, personnel sensors, air systems, and any other device or sensor known in the art. In addition, the antenna 603 may transmit signals to any of the aforementioned devices, systems, and sensors, as well as any other device or sensor known in the art. Although the antenna 603 is used for wireless communications, the central station 601 may be connected to any number of sensors or devices using cables, lines, wires, or by any other technique known in the art.

In one or more embodiments, monitoring devices at a worksite may transmit data directly to one or more monitoring devices 605. The monitoring devices 605 may include any one of a screen (e.g., a liquid crystal display (LCD), a plasma display, touchscreen, cathode ray tube (CRT) monitor, projector, 2D display, 3D display, or other display device), a printer, external storage, or any other output device. The monitoring devices 605 may display measurements from any number of sensors or signals sent from devices at worksite. The monitoring devices 605 may display live video from a confined workspace, or any other workspace described herein, for example.

As shown in FIG. 6B, a monitoring device 605 is includes a graphical user interface (GUI) 606 having measurements and data viewable and controllable using the GUI 606. The GUI 606 may include one or more buttons (e.g., radio buttons), data fields (e.g., input fields), banners, menus (e.g., user input menus), boxes (e.g., input or output text boxes), tables (e.g., data summary tables), sections (e.g., informational sections or sections capable of minimizing/maximizing), screens (e.g., welcome screen or home screen), and/or user selection menus (e.g., drop down menus). In addition, the GUI 606 may include one or more separate interfaces and may be usable in a web browser or as a standalone application. Using the GUI 606, a user may monitor data from any one of the sensors, devices, and system described herein and may control a number of different parameters for each of the sensors, devices, and systems displayed on the GUI 606.

In addition, the central station may include a compliance monitor 607. The compliance monitor 607 may be configured to obtain or receive signals from sensors, devices, or other equipment related to compliance regulations. Many countries require specific rules and regulations to be complied with when performing particular operations or in certain environments. As such, the compliance monitor 607 may monitor worksite compliances and compare the worksite compliances with local, state, and federal regulations. Worksite compliances may include breathing air quality, operability and condition of electrical equipment, emergency service stations, such as eyewash stations and/or showers, elevator, ladder, and bicycle maintenance, fire safety standards, and calibration requirements and status of components and tools used at the worksite, among many others. For example, compliance monitor 607 may be connected to a network or a server containing local, state, and federal regulations related to emergency services, such as eyewash stations. If the eyewash station is in use or has been used within a predetermined time, the compliance monitor 607 may then indicate that the eyewash station is not in compliance.

In one or more embodiments, the central station 601 may include one or more recording devices 609. The recording devices 609 may include one or more storage devices (e.g., a hard disk, an optical drive such as a compact disk (CD) drive or digital versatile disk (DVD) drive, a flash memory stick, etc.) and/or memory (e.g., random access memory (RAM), cache memory, flash memory, etc.). The recording devices 609 (and/or any information stored therein) may be a data store such as a database, a file system, one or more data structures (e.g., arrays, link lists, tables, hierarchical data structures, etc.) configured in a memory, an extensible markup language (XML) file, any other suitable medium for storing data, or any suitable combination thereof. The recording devices 609 may be a device internal to the central station 601. Alternatively, the recording devices 609 may be an external storage device operatively connected to the central station 601.

The recording devices 609 may be configured to store and/or log data from any one of the sensors, devices, or system discussed herein, or any other known in the art. The recording devices 609 may be accessible by the monitoring devices 605 and/or the compliance monitor 607.

Further, the central station 601 may include a control device 611. Referring now to FIG. 6C, the control device 611 may include one or more input device(s) 613, such as a touchscreen, keyboard, mouse, microphone, touchpad, electronic pen, or any other type of input device. The control device 611 may also include a computing device 615 having one or more computer processor(s) 617, memory 619 and a GUI 621 for communicating with one or more input devices 613. The computer processor(s) 617 may be an integrated circuit for processing instructions. For example, the computing processor(s) may be one or more cores, or micro-cores of a processor. The computing device 615 may be communicatively coupled to a network 623 (e.g., a local area network (LAN), a wide area network (WAN) such as the Internet, mobile network, or any other type of network) through wires, cables, fibers, optical connectors, a wireless connection, or a network interface connection (not shown).

The input device(s) may be locally or remotely (e.g., via the network 623) connected to the computer processor(s) 617, memory 619, and GUI 621. Many different types of systems exist, and the aforementioned input and monitoring device(s) may take other fauns.

Further, one or more elements of the control device 611 may be located at a remote location and connected to the other elements over a network 623. Further, embodiments of the disclosure may be implemented on a distributed system having a plurality of nodes, where each portion of the disclosure may be located on a different node within the distributed system. In one embodiment of the disclosure, the node corresponds to a distinct computing device. Alternatively, the node may correspond to a computer processor with associated physical memory. The node may alternatively correspond to a computer processor or micro-core of a computer processor with shared memory and/or resources.

In one or more embodiments, the control station may generate reports based on data received and/or transmitted at a worksite. In particular, control device 611 may communicate with one or more reporting devices 625. The reporting device may be configured to access recording devices 609 and monitoring devices 605 to collect data and generate reports. Reports can include the number of dangerous areas in a facility or at a worksite. Using the reports, the reporting device 625 may also calculate an estimated time for repair or cleanup of an emergency situation based on previous occurrences, for example. The reporting device 625 may also generate evacuation plans based on the number of people working and their locations. Further, reporting device 625 may also determine number of hours worked for a given number of employees and which may then be used to determine what behaviors lead to injury, or may be used for lesson learning and to prevent future injury.

A safety control system in accordance with one or more embodiments may efficiently, accurately, and quickly monitor a number of safety hazards, working conditions, health status of personnel, emergency procedures, and many others. Using monitored data, systems, devices, and/or components may be controlled at a worksite to mitigate and/or prevent hazardous working environments, and ultimately, injury and/or death of personnel. Further, reports may be generated to be analyzed and/or used to further prevent injury during given operations and at particular worksites.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

What is claimed is:

1. A safety control system for a worksite comprising:
a video system comprising a video camera communicable with a video monitoring device and a video recording device;
a personnel system comprising a personnel sensor communicable with a personnel monitoring device and a personnel recording device;
a component system comprising a component sensor communicable with a component monitoring device and a component recording device; and
a reporting system configured to access at least one of the video recording device, the personnel recording device, and the component recording device,
wherein the reporting system generates a report including information from at least one of the video recording device, the personnel recording device, and the component recording device.

2. The safety control system of claim 1, wherein the video system is configured to monitor and record data for at least one of a worksite perimeter, a confined workspace, a worksite task, and a component at the worksite.

3. The safety control system of claim 1, wherein the personnel system is configured to monitor and record data of at least one of personnel health, personnel training, personnel equipment, and personnel biometrics.

4. The safety control system of claim 1, wherein the personnel system further comprises a personnel video device communicable with the personnel monitoring device and personnel recording device.

5. The safety control system of claim 4, wherein the personnel video device is configured to feed video to at least one of the personnel monitoring device and the personnel recording device in real time.

6. The safety control system of claim 1, further comprising a central monitoring system communicable with each of the video monitoring device, the personnel monitoring device, and the component monitoring device.

7. The safety control system of claim 1, wherein the component system is configured to monitor and record data of at least one of a gas system, an air delivery system, worksite equipment, a confined space system, and a cleaning system.

8. The safety control system of claim 1, wherein the safety control system is a mobile system.

9. A personnel monitoring system comprising:
a personnel sensor configured to detect attributes of a user,
wherein the personnel sensor is communicable with a personnel monitoring device and a personnel recording device, and
wherein the attributes of the worker comprise at least one of biometrics, ambient temperature, breathing air, surrounding gases, training, and fatigue.

10. The personnel monitoring system of claim 9, wherein the personnel monitoring system is configured to control access of the user based on the attributes of the user.

11. The personnel monitoring system of claim 9, wherein, when the training of the user is inadequate, access to a given worksite by the user is denied by at least one of the personnel monitoring system and an access control system.

12. The personnel monitoring system of claim 11, wherein the access control system is specific to the given worksite.

13. The personnel monitoring system of claim 9, wherein, when at least one of the temperature of the worker, the heart-rate of the worker, and the fatigue of the worker exceeds a given threshold, an alarm is triggered by the personnel sensor.

14. The personnel monitoring system of claim 9, wherein, when the breathing air of the worker is below a given threshold, an alarm is triggered by the personnel sensor.

15. The personnel monitoring system of claim 9, wherein, when the surrounding gases of the worker contain a given percentage of a noxious gas, an alarm is triggered by the personnel sensor.

16. The personnel monitoring system of claim 9, further comprising a personnel indicator accessible by the personnel monitoring device indicating a status of the worker at the worksite.

17. The personnel monitoring system of claim 15, wherein the status of the worker at the worksite is indicated on the personnel monitoring device based on a color scheme.

18. A compliance monitoring system comprising:
a compliance sensor communicable with a compliance monitoring device and a compliance recording device,
wherein the compliance monitoring system is configured to compare data from the compliance sensor with compliance requirements, and
wherein the compliance requirements are worksite specific.

19. The compliance monitoring system of claim 17, wherein the compliance sensor is configured to measure at least one of fire, air, equipment, and calibration data of a worksite and compare the measured data with compliance requirements.

20. The compliance monitoring system of claim 18, wherein the equipment comprises at least one of a crane, lift, elevator, hose, ladder, bicycle, electrical equipment, operating equipment, and safety equipment.

21. The compliance monitoring system of claim 18, wherein the calibration data comprises calibration of at least one of operating equipment, safety equipment, worksite tools, air delivery, and worksite gases.

22. An air system for producing breathing air comprising:
an air compressor in fluid communication with a wet tank;
a dry tank in fluid communication with the wet tank and a collection pot; and
a collection pot in fluid communication with a manifold,
wherein the manifold is configured to output breathing air through an outlet to at least one end user.

23. The air system of claim 22, further comprising:
an air quality sensor configured to measure quality of breathing air produced by the air system;
an air intake sensor configured to measure quality of intake air at the air compressor;
a flowmeter configured to measure a flow rate of the breathing air produced by the air system;
a pressure meter configured to measure a pressure of the breathing air produced by the air system; and
a control system configured to control production of breathing air based on at least one of the air quality sensor, air intake sensor, flowmeter, and pressure meter.

24. The air system of claim 23, wherein the control system is further configured to control output breathing air from the manifold.

25. The air system of claim 23, wherein the air quality sensor is configured to alarm when the quality of breathing air falls outside a predetermined range.
